(19) United States
(12) Patent Application Publication
(10) Pub. No.: US 2021/0393920 A1
(43) Pub. Date: Dec. 23, 2021

(54) CATHETER ASSEMBLY

(71) Applicant: TERUMO KABUSHIKI KAISHA, Tokyo (JP)

(72) Inventors: Shota YAMAMOTO, Mitaka-shi (JP); Ryoji KOBAYASHI, Hadano-shi (JP)

(73) Assignee: TERUMO KABUSHIKI KAISHA, Tokyo (JP)

(21) Appl. No.: 17/465,467

(22) Filed: Sep. 2, 2021

Related U.S. Application Data
(63) Continuation of application No. PCT/JP2020/010685, filed on Mar. 12, 2020.

(30) Foreign Application Priority Data
Mar. 18, 2019 (JP) 2019-049385

(51) Int. Cl. A61M 25/00; A61M 25/06; A61M 39/26; A61M 39/06

(52) U.S. Cl. CPC A61M 25/0097; A61M 25/06; A61M 2039/062; A61M 39/06; A61M 39/26

(57) ABSTRACT

A catheter assembly includes: a catheter; a catheter hub provided at a proximal end of the catheter; a valve body fixed in the catheter hub; and an opening member housed in the catheter hub to open the valve body. The opening member is movable between a first position proximal of the valve body and a second position for opening the valve body by being advanced by a connector inserted into the catheter hub. The opening member includes an engaging portion that engages with the connector with movement to the second position caused by the connector, and maintains the engagement with the connector and retracts the opening member from the valve body to the first position when the connector is retracted in a proximal direction and detached from the catheter hub.

### CROSS-REFERENCE TO RELATED APPLICATIONS

This application is a bypass continuation of PCT Application No. PCT/JP2020/010685, filed on Mar. 12, 2020, which claims priority to Japanese Application No. 2019-049385, filed on Mar. 18, 2019. The contents of these applications are hereby incorporated by reference in their entireties.

### BACKGROUND

The present disclosure relates to a catheter assembly including a valve body that inhibits blood from flowing out and an opening member that opens the valve body.

A catheter assembly that functions as an indwelling needle is used when performing an infusion and a blood transfusion. For example, a catheter assembly disclosed in JP 2014-528807 A includes a catheter and a catheter hub (catheter adapter) fixed to the catheter. The catheter assembly is punctured into a patient with an introducer needle housed in the catheter during use, and the introducer needle is detached from the catheter and catheter hub after being punctured. Then, the catheter assembly serves as an inlet/outlet for a medicinal liquid or blood as a connector of a medical device (infusion or blood transfusion tube) is inserted into the catheter hub after detachment.

In addition, the catheter assembly disclosed in JP 2014-528807 A includes a valve body (septum) that inhibits blood leakage during indwelling, and an opening member (actuator) configured to open the valve body, in the catheter hub. The opening member is pushed out by the connector inserted into the catheter hub to open a slit of the valve body such that a liquid can flow.

### SUMMARY

An opening state of the valve body is continued after the opening member once opens the slit of the valve body in the catheter assembly disclosed in JP 2014-528807 A. Therefore, blood, an infusion solution, or the like leaks from a proximal end of the catheter hub after detaching the connector from the catheter hub.

A mechanism for pulling out the opening member from the valve body at the time of detaching the connector is also conceivable. Examples of this type of mechanism include a valve body having a high repulsive force and a spring that biases the opening member in the proximal direction. However, this type of mechanism causes another inconvenience, which is difficulty in pushing the opening member into the valve body when the connector has been inserted.

Certain embodiments of the present disclosure have been developed to solve the above problems, and an object thereof is to provide a catheter assembly capable of inhibiting leakage of a liquid by opening a valve body with insertion of a connector without requiring a large operating force and closing the valve body with detachment of the connector.

According to one embodiment, a catheter assembly includes: a catheter; a catheter hub provided at a proximal end of the catheter; a valve body fixed in the catheter hub; and an opening member housed in the catheter hub to open the valve body. The opening member is movable between a first position closer to a proximal side than the valve body and a second position for opening the valve body by being advanced by a connector inserted into the catheter hub. The opening member has an engaging portion that engages with the connector with movement to the second position caused by the connector, and maintains the engagement with the connector and retracts the opening member from the valve body to the first position when the connector is retracted in a proximal direction and detached from the catheter hub.

In the above catheter assembly, the opening member is provided with the engaging portion, so that the opening member and the connector are easily engaged as the connector advances. That is, the engaging portion can open the valve body by integrating the opening member and the connector without requiring a large operating force. In addition, the catheter assembly can retract the opening member following the connector with the engagement of the engaging portion at the time of detaching the connector. As a result, the valve body can be smoothly closed from an open state and the leakage of the liquid can be suppressed.

### BRIEF DESCRIPTION OF THE DRAWINGS

FIG. 1 is a perspective view illustrating an overall configuration of a catheter assembly according to a first embodiment of the present invention;

FIG. 2 is a side cross-sectional view illustrating the inside of a catheter hub when an opening member is located at a first position;

FIG. 3A is an explanatory view schematically illustrating an engagement state between the opening member and a connector according to a first configuration example of the first embodiment, FIG. 3B is an explanatory view schematically illustrating an engagement state between the opening member and the connector according to a second configuration example of the first embodiment, and FIG. 3C is an explanatory view schematically illustrating an engagement state between the opening member and the connector according to a third configuration example of the first embodiment;

FIG. 4A is a first explanatory view illustrating an operation of the opening member at the time of inserting the connector, and FIG. 4B is a second explanatory view illustrating the operation of the opening member at the time of inserting the connector;

FIG. 5A is a first explanatory view schematically illustrating an engagement state between an opening member of a catheter assembly and a connector according to a second embodiment of the present invention, FIG. 5B is a second explanatory view schematically illustrating the engagement state between the opening member and the connector, which is subsequent to FIG. 5A, and FIG. 5C is a third explanatory view schematically illustrating the engagement state between the opening member and the connector, which is subsequent to FIG. 5B;

FIG. 6A is a first side cross-sectional view illustrating an opening member and a connector in a first configuration example of a catheter assembly according to a third embodiment, and FIG. 6B is a second side cross-sectional view illustrating an engagement state between the opening member and the connector, which is subsequent to FIG. 6A; and

FIG. 7A is an explanatory view schematically illustrating the engagement state between the opening member and the connector according to a second configuration example of the third embodiment, FIG. 7B is an explanatory view schematically illustrating the engagement state between the opening member and the connector according to a third configuration example of the third embodiment, and FIG. 7C is an explanatory view schematically illustrating the engagement state between the opening member and the connector according to a fourth configuration example of the third embodiment.

### DETAILED DESCRIPTION

Hereinafter, embodiments of the present invention will be described in detail with reference to the accompanying drawings.

### First Embodiment

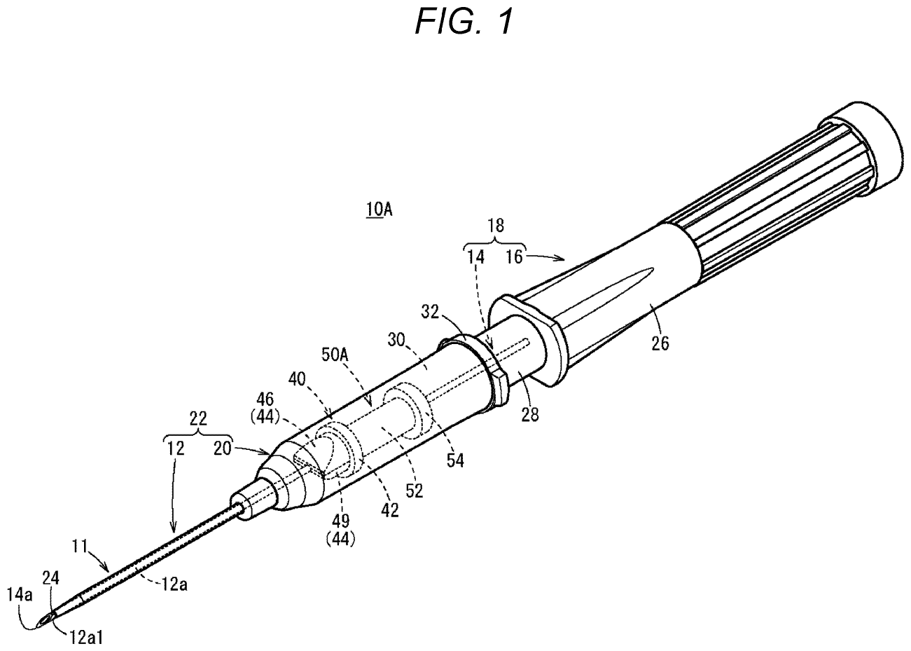

A catheter assembly 10A according to a first embodiment of the present invention has a catheter 12 that is inserted to indwell inside a patient's body (living body) as illustrated in FIG. 1, and is used to construct an inlet/outlet for a liquid (a medicinal liquid and blood) during an infusion, a blood transfusion, or the like. The catheter 12 is configured as a peripheral venous catheter. The catheter 12 may be a catheter longer than a peripheral venous catheter (for example, a central venous catheter, PICC, midline catheter, and the like). In addition, the catheter 12 is not limited to a venous catheter, and may be configured as an arterial catheter such as a peripheral arterial catheter.

As illustrated in FIG. 1, the catheter assembly 10A has an operating body 18 formed of an inner needle 14 and a needle hub 16 fixed to a proximal end of the inner needle 14. In addition, the catheter assembly 10A has a catheter indwelling body 22 configured of the above-described catheter 12 and a catheter hub 20 fixed to a proximal end of the catheter 12.

In the catheter assembly 10A, a multi-structure needle 11 in which the inner needle 14 is inserted into the catheter 12 is formed as the operating body 18 is assembled to a proximal end of the catheter indwelling body 22 in an initial state before use (product provided state). In the multi-structure needle 11, the needle tip 14a of the inner needle 14 protrudes from a distal end of the catheter 12, and the inner needle 14 and the catheter 12 can be integrally punctured into the patient.

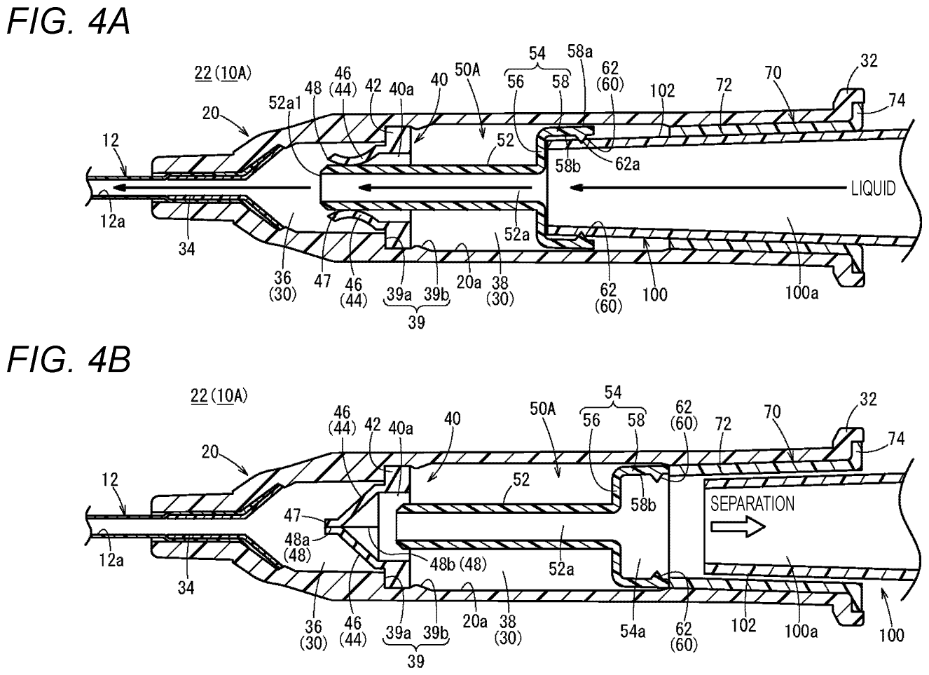

When using the catheter assembly 10A, a user, such as a doctor and a nurse, grips and operates the needle hub 16 to puncture the multi-structure needle 11 into the patient's body, thereby setting a puncture state in which the needle tip 14a reaches a blood vessel. Further, the user inserts the catheter 12 into the blood vessel by advancing the catheter 12 relative to the inner needle 14 while maintaining the puncture state. Then, the catheter indwelling body 22 indwells in the patient by retracting the inner needle 14 relative to the catheter 12 and further removing the inner needle 14 from the catheter hub 20. Then, the catheter indwelling body 22 can perform treatment such as administration of a medicinal liquid or blood to a patient and blood collection from the patient by connecting a connector 100 of a medical device (see FIG. 4) to the catheter hub 20. Hereinafter, each configuration of the catheter assembly 10A will be described in detail.

The inner needle 14 of the catheter assembly 10A (the operating body 18) is configured as a hollow tube (or a solid rod) having rigidity capable of puncturing a skin of a living body, and has the sharp needle tip 14a at a distal end thereof. An outer peripheral surface of the inner needle 14 is provided with a groove 24 for flashback that guides blood to the proximal side when puncturing the blood vessel. Incidentally, the structure for performing flashback is not particularly limited, and for example, a configuration having a hole (not illustrated) communicating with an inner space of the inner needle 14 on the proximal side of the distal end of the catheter 12 may be provided. The catheter assembly 10A has a needle puncture deterrence structure that inhibits erroneous puncture of the needle tip 14a after puncturing the inner needle 14 (a cover that covers the needle tip 14a after puncture, a blunt needle protruding from the needle tip 14a, or the like).

Examples of a constituent material of the inner needle 14 include a metal material such as stainless steel, aluminum or an aluminum alloy, and titanium or a titanium alloy, a hard resin, ceramics, and the like. The inner needle 14 is firmly fixed to the needle hub 16 by an appropriate fixing means such as fusion, adhesion, and insert molding.

The needle hub 16 forms a grip portion to be gripped by the user in the initial state in which the catheter indwelling body 22 and the operating body 18 are assembled. The needle hub 16 includes a hub main body 26 gripped by the user and an inner needle support portion 28 integrally molded at a distal end of the hub main body 26. The hub main body 26 is formed into a cylindrical shape on the proximal side, and is gradually deformed into a square tube shape toward the distal side. The inner needle support portion 28 is formed into a columnar shape protruding from the hub main body 26 in the distal direction, and holds a proximal portion of the inner needle 14 at the central portion thereof.

A constituent material of the needle hub 16 is not particularly limited, but a thermoplastic resin, such as polypropylene, polycarbonate, polyamide, polysulfone, polyarylate, and a methacrylate-butylene-styrene copolymer can be applied.

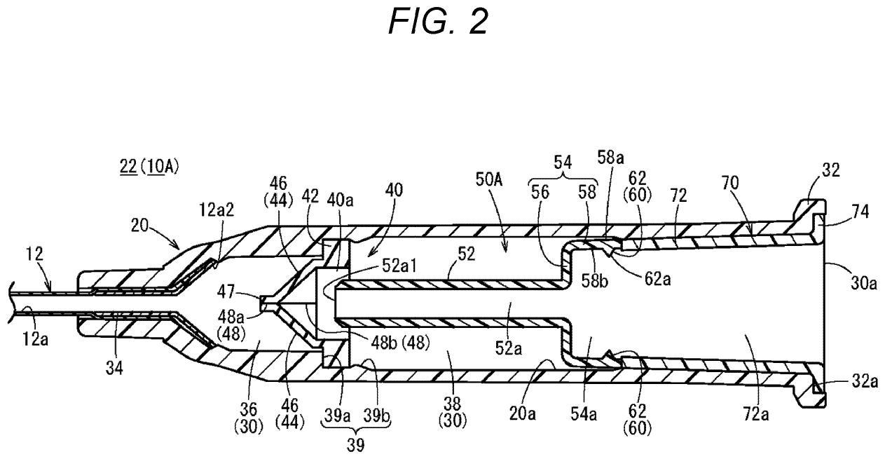

On the other hand, the catheter 12 of the catheter assembly 10A is configured as a flexible hollow tube in which a lumen 12a is formed inside. An outer shape of the catheter 12 and the lumen 12a are formed into a perfect circular shape in a cross-sectional view orthogonal to the axial direction, and extend along the axial direction of the catheter 12. The lumen 12a communicates with a distal opening 12a1 formed at a distal end of the catheter 12 and a proximal opening 12a2 (see FIG. 2) formed at a proximal end of the catheter 12.

A material configuring the catheter 12 is not particularly limited, but a transparent soft resin material may be applied. Examples of a constituent material of the catheter 12 include a fluorine-based resin such as polytetrafluoroethylene (PTFE), an ethylene-tetrafluoroethylene copolymer (ETFE), and a perfluoroalkoxy fluorine resin (PFA), an olefin-based resin such as polyethylene and polypropylene or a mixture thereof, polyurethane, polyester, polyamide, a polyether nylon resin, a mixture of the olefin-based resin and an ethylene-vinyl acetate copolymer, and the like.

A length of the catheter 12 is not particularly limited, and can be appropriately designed according to the application, various conditions, and the like, and is set to, for example, about 14 to 500 mm. The proximal end of the catheter 12 is inserted and fixed inside the catheter hub 20.

The catheter hub 20 is exposed on the patient's skin in a state in which the catheter 12 has been inserted into the patient's blood vessel, and indwells together with the catheter 12 by being pasted with a tape or the like. A material forming the catheter hub 20 is not particularly limited, but, for example, the materials exemplified in the needle hub 16 may be appropriately adopted.

As illustrated in FIGS. 1 and 2, the catheter hub 20 is formed into a cylindrical shape tapered in the distal direction. A hollow portion 30 is provided inside the catheter hub 20. The hollow portion 30 holds the catheter 12 on the distal side and communicates with the lumen 12a (proximal opening 12a2). In addition, the hollow portion 30 communicates with the proximal opening portion 30a of the catheter hub 20 on the proximal side. A flange 32, which protrudes to the radially outer side and extends along the circumferential direction, is provided on the outer peripheral surface of the catheter hub 20 on the proximal side.

The catheter 12 and the catheter hub 20 are fixed by an appropriate fixing means such as caulking, fusion, or adhesion. In FIG. 2, a caulking pin 34 is inserted into the hollow portion 30 of the catheter hub 20 to caulk the caulking pin 34 by sandwiching the catheter 12 between an inner wall 20a of the catheter hub 20 and the caulking pin 34, thereby fixing the catheter 12.

A valve body 40, an opening member 50A, and a fixing member 70 are housed in the catheter hub 20. The valve body 40 is configured as a hemostasis valve that divides the hollow portion 30 into a distal-side space 36 and a proximal-side space 38 and blocks blood flowing from the lumen 12a of the catheter 12 into the hollow portion 30 from leaking from the proximal opening portion 30a. An example of the valve body 40 is an application of a duckbill valve.

For example, the valve body 40 has an annular portion 42 fixed to the inner wall 20a of the catheter hub 20 and a valve main body 44 protruding from the annular portion 42 in the distal direction. The annular portion 42 is configured as a block that protrudes to the radially outer side of the valve main body 44 and has a sufficient thickness in the axial direction of the catheter hub 20.

The inner wall 20a of the catheter hub 20 is provided with a fixing structure 39, which inhibits the annular portion 42 from moving in the axial direction in a state in which the valve body 40 is arranged, in order to fix the annular portion 42. The fixing structure 39 is configured of a stepped portion 39a in which the diameter of the hollow portion 30 is slightly reduced, and a locking convex portion 39b protruding inward at a position away from the stepped portion 39a toward the proximal side.

The valve main body 44 is formed into a cylindrical shape on the proximal side continuous with the annular portion 42 and formed into a tubular shape having a pair of inclined portions 46 that become close to each other in the distal direction. A cylindrical side portion 49 (see FIG. 1) is formed in a circumferentially adjacent part of the pair of inclined portions 46. A valve space 40a that narrows in the distal direction is formed inside the valve body 40.

A distal end of the pair of inclined portions 46 forms an extending end 47 extending in the width direction. The slit 48 of the valve body 40 is configured of a front slit 48a, formed along the longitudinal direction of the extending end 47, and side slits 48b, formed in the cylindrical side portion 49 configured between the pair of inclined portions 46 from both ends of the extending end 47, when viewed from the front. The front slit 48a and the side slit 48b are continuous. The side slits 48b extend to be parallel in the proximal direction from the extending end 47 to a distal end of the annular portion 42.

The annular portion 42, the pair of inclined portions 46, and the side portion 49 are formed in a shape that allows insertion of the inner needle 14 through the slit 48 in the initial state of the catheter assembly 10A and self-closes the slit 48 with the removal of the inner needle 14 from the valve body 40. In addition, the pair of inclined portions 46 are greatly separated from each other by inserting the opening member 50A arranged on the proximal side of the valve body 40 to open the slit 48 (see also FIG. 4). Incidentally, the configuration of the valve body 40 is not limited to the duckbill valve, and various configurations can be adopted. For example, the valve body 40 may be a disk valve having a flat film.

In the initial state, the opening member 50A is arranged closer to the proximal side than the valve main body 44 in the hollow portion 30. Specifically, the opening member 50A is located in the proximal-side space 38 inside the catheter hub 20 at an initial position on the distal side of the fixing member 70.

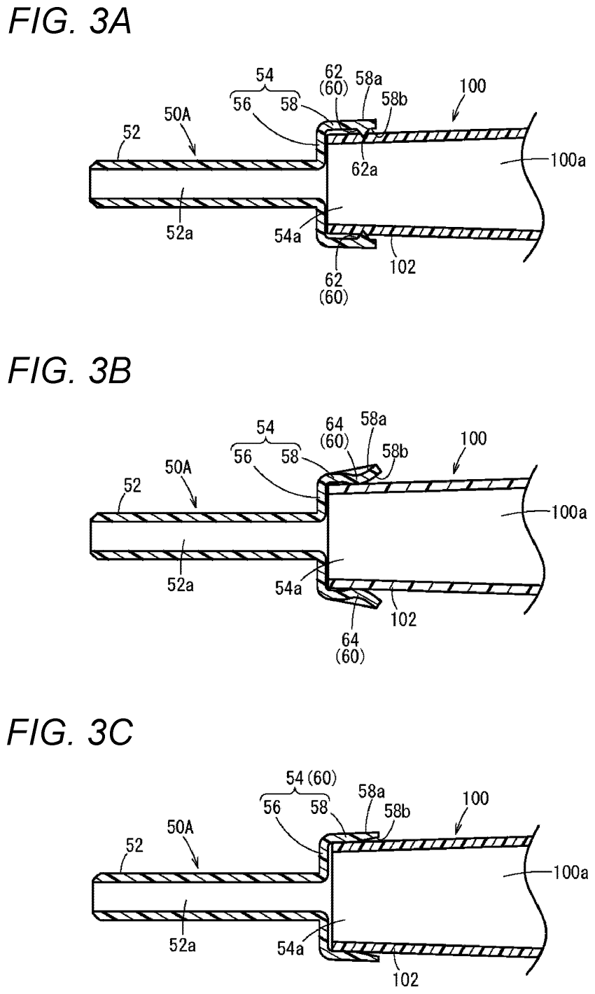

The opening member 50A has a cylindrical nozzle portion 52 and a proximal tubular portion 54 that is continuous with a proximal end of the nozzle portion 52 and is formed to have a larger diameter than the nozzle portion 52. A flow passage 52a enabling a liquid to flow therethrough is formed inside the nozzle portion 52. A space portion 54a, which communicates with the flow passage 52a and allows insertion of the connector 100 of the medical device (see FIG. 3A), is formed inside the proximal tubular portion 54.

When the opening member 50A moves in the distal direction relative to the valve body 40, the nozzle portion 52 penetrates the slit 48 of the valve body 40 and pushes and widen the valve main body 44 (the pair of inclined portions 46). The nozzle portion 52 is formed into a cylindrical shape having a diameter sufficiently smaller than a diameter of the hollow portion 30 of the catheter hub 20. The nozzle portion 52 extends along the axial direction of the nozzle portion 52 with constant outer diameter and inner diameter. Incidentally, the nozzle portion 52 may be formed into a tapered shape or the like that is gradually tapered in the distal direction.

The flow passage 52a of the nozzle portion 52 extends along the axial direction of the nozzle portion 52, and has a distal end communicating with a distal opening 52a1 formed at a distal end of the nozzle portion 52. The distal side of the nozzle portion 52 is arranged inside the annular portion 42 of the valve body 40 (valve space 40a) in the initial state. In addition, a tapered surface whose diameter decreases in the distal direction is formed on an outer peripheral surface of the nozzle portion 52 on the distal side.

On the other hand, the proximal tubular portion 54 provided at the proximal end of the nozzle portion 52 has a cylindrical shape extending a short length in the axial direction of the opening member 50A. An axial length of the proximal tubular portion 54 is sufficiently shorter than an axial length of the nozzle portion 52. For example, the axial length of the proximal tubular portion 54 is preferably set to ½ or less of the axial length of the nozzle portion 52.

Specifically, the proximal tubular portion 54 is configured of a disc-shaped end wall portion 56 extending from the proximal end of the nozzle portion 52 to the radially outer side, and a side wall portion 58 extending from an outer edge of the end wall portion 56 in the proximal direction. The end wall portion 56 forms a bottom that closes a distal part of the proximal tubular portion 54, and has distal surface and proximal surface that are flat.

The side wall portion 58 is formed into a tapered shape whose diameter gradually increases from the outer edge of the end wall portion 56 in the proximal direction. A diameter of an outer peripheral surface 58a of the side wall portion 58 is slightly smaller than a diameter of the inner wall 20a forming the proximal-side space 38, and substantially coincides with a diameter of an outer peripheral surface on the distal side of the fixing member 70. As a result, the side wall portion 58 moves to a position close to the inner wall 20a when the opening member 50A is displaced. In addition, the proximal tubular portion 54 interferes with the fixing member 70 in the housed state of the catheter hub 20, so that the opening member 50A is inhibited from being detached from the proximal direction.

A diameter of an inner peripheral surface 58b (the space portion 54a) of the side wall portion 58 is larger than a diameter of an inner peripheral surface on the distal side of the fixing member 70 (or substantially coincides with the diameter of the inner peripheral surface). In addition, the diameter of the inner peripheral surface 58b is set to be slightly larger than the diameter (outer diameter) of a distal-side outer peripheral surface 102 (see FIG. 3A) of the connector 100.

The opening member 50A is provided with an engaging portion 60, which engages with the connector 100, on the proximal tubular portion 54 (side wall portion 58) as illustrated in FIGS. 2 and 3A. As a first configuration example of the present embodiment, a plurality of protrusions 62 protruding from the inner peripheral surface 58b to the radially inner side are applied as the engaging portion 60. The plurality of protrusions 62 are provided at equal intervals along the circumferential direction on the proximal side of the side wall portion 58.

Each of the protrusions 62 is formed into a triangular shape having a top 62a in a side cross-sectional view along the axial direction of the opening member 50A, and is configured such that the top 62a bites (forms an anchor) into the distal-side outer peripheral surface 102 of the connector 100 as the connector 100 is inserted. The amount of each of the protrusions 62 (top 62a) protruding from the inner peripheral surface 58b is small with respect to the diameter of the inner peripheral surface 58b. For example, each of the tops 62a is formed on the side wall portion 58 so as to be located on the inner side within a range of about 0.01 mm to 1 mm with respect to the diameter of the distal-side outer peripheral surface 102 of the connector 100.

Incidentally, it is sufficient to provide one or more protrusions 62 on the side wall portion 58, or the protrusions 62 may be formed into a ring shape that is continuous in a series along the circumferential direction of the side wall portion 58. Further, a cross-sectional shape of the protrusion 62 is not particularly limited, and may be formed into a right triangle whose proximal side is inclined and whose distal side is perpendicular to the inner peripheral surface 58b, or may be formed into a quadrangle or a semicircle.

In addition, the configuration of the engaging portion 60 that engages with the connector 100 is not limited to the protrusion 62, and various configurations may be adopted. For example, as in a second configuration example illustrated in FIG. 3B, the engaging portion 60 may be formed by applying one or more elastic pieces 64 that elastically protrude to the radially inner side of the side wall portion 58. The elastic piece 64 is formed into an arc shape that curves radially inward from a connecting part at a distal end in the proximal direction and extends radially outward from a valley part in the middle. The elastic piece 64 engages with the connector 100 by generating an appropriate frictional force between an inner surface of the valley part and the distal-side outer peripheral surface 102 of the connector 100.

In addition, for example, as in a third configuration example illustrated in FIG. 3C, the engaging portion 60 may be configured to fit with the distal-side outer peripheral surface 102 by appropriately designing the shape (diameter) of the inner peripheral surface 58b of the side wall portion 58 according to the diameter of the distal-side outer peripheral surface 102 of the connector 100. In this case, a proximal end of the inner peripheral surface 58b is preferably formed as a tapered surface such that the connector 100 can be easily inserted. Incidentally, various configurations (coating, roughening of the inner peripheral surface 58b, or the like) for improving the frictional resistance with the distal-side outer peripheral surface 102 may be applied to the side wall portion 58.

Alternatively, a configuration in which the engaging portion 60 is suctioned to the distal-side outer peripheral surface 102 (a sucker or the like) and a configuration in which the engaging portion 60 is detachably adhered to the distal-side outer peripheral surface 102 (an adhesive or the like) can be applied, although not illustrated. In this manner, the configuration in which the engaging portion 60 engages with the connector 100 by biting, frictional force, suction, adhesion, or the like does not change a conventional configuration of the connector 100, which has an advantage that it is easy to use as the engaging portion 60.

Incidentally, a material forming the opening member 50A is not particularly limited, but an appropriate material may be applied according to a type of the engaging portion 60 described above, and, for example, the material or the like described in the needle hub 16 can be appropriately selected. As an example, a material harder than the connector 100 may be applied in the case of the configuration in which the engaging portion 60 bites into the connector 100 (the protrusion 62) or the configuration in which the engaging portion 60 fits with the connector 100 by the frictional force. In addition, a material more flexible than the connector 100 may be applied in the case of the configuration in which the engaging portion 60 engages with the connector by the frictional force during elastic deformation, adsorption, adhesion, or the like.

Returning to FIG. 2, the fixing member 70 is provided on the proximal side of the catheter hub 20 and functions as a regulating portion that regulates detachment of the opening member 50A. The fixing member 70 is formed into a cylindrical shape, and is fixed to the catheter hub 20 by fitting with the inner wall 20a of the catheter hub 20 in a state in which the opening member 50A is housed in the hollow portion 30. Incidentally, a fixing means between the catheter hub 20 and the fixing member 70 is not particularly limited, and adhesion, fusion, or the like may be used.

The fixing member 70 includes a fixed tubular body 72 having a through-hole 72a and an annular convex portion 74 provided at a proximal end of the fixed tubular body 72. The proximal end of the fixed tubular body 72 forms the proximal opening portion 30a of the catheter hub 20. The annular convex portion 74 is arranged in an annular groove portion 32a inside a flange 32 formed at the proximal end of the catheter hub 20 to regulate the displacement of the fixing member 70 in the distal direction. For the opening member 50A and the fixing member 70, a structure that regulates the rotation of the opening member 50A in the circumferential direction relative to the catheter hub 20 (for example, a structure in which a protruding piece (not illustrated) is provided on one side and a concave portion (not illustrated) capable of housing a protruding piece is provided on the other side) may be applied.

The catheter assembly 10A according to the present embodiment is basically configured as described above, and operations thereof will be described hereinafter.

As described above, the catheter assembly 10A is used at the time of constructing the inlet/outlet for the infusion, the blood transfusion, the blood sampling, and the like to the patient. The user grips and operates the needle hub 16 of the catheter assembly 10A in the initial state shown in FIG. 1 to puncture the patient with the multi-structure needle 11.

When the needle tip 14a of the inner needle 14 reaches the blood vessel, blood flows through the groove 24 of the inner needle 14 into the lumen 12a of the catheter 12. The user visually confirms this flashback of blood to confirm that the catheter 12 has secured the blood vessel. The flashback blood flows from the proximal opening 12a2 of the catheter 12 into the distal-side space 36 of the catheter hub 20, but the blood is inhibited from flowing into the proximal-side space 38 as the valve body 40 is closed due to the opening member 50A located at the initial position.

In the puncture state, the user advances the catheter 12 relative to the inner needle 14 to insert the catheter 12 into the blood vessel, and retracts the inner needle 14 with respect to the catheter 12 when the catheter 12 is inserted into the blood vessel to some extent. As a result, the inner needle 14 is detached from the proximal opening portion 30a of the catheter hub 20, the operating body 18 is separated from the catheter indwelling body 22, and the catheter indwelling body 22 is indwelled in the patient. In addition, when the needle tip 14a of the inner needle 14 is detached from the valve body 40 at the time of retracting the inner needle 14, the slit 48 is closed. The state of the catheter indwelling body 22 at this time is illustrated in FIG. 2, and the position of the opening member 50A is set as a first position.

In the indwelling state of the catheter indwelling body 22, the user inserts the connector 100 of the medical device (a tube of an infusion line or a blood transfusion line, a syringe, or the like) from the proximal opening portion 30a of the catheter hub 20 into the hollow portion 30 as illustrated in FIGS. 2 and 4A. When the connector 100 moves in the distal direction inside the proximal-side space 38, its distal surface reaches the proximal tubular portion 54 of the opening member 50A.

When the connector 100 comes into contact with the protrusion 62 (engaging portion 60) of the proximal tubular portion 54, the opening member 50A at the first position is pushed out in the distal direction. The opening member 50A receives some resistance from the inner wall 20a of the catheter hub 20 and the valve body 40 when displaced in the distal direction. As a result, the distal-side outer peripheral surface 102 of the connector 100 passes over the protrusion 62 and advances in the distal direction inside the space portion 54a of the proximal tubular portion 54. The protrusion 62 strongly bites into the distal-side outer peripheral surface 102 formed into a tapered shape at an appropriate position, thereby forming the anchor. As a result, the state in which the opening member 50A and the connector 100 are engaged is achieved.

Then, the opening member 50A moves inside the valve body 40 as the connector 100 is pushed in, and the nozzle portion 52 separates the pair of inclined portions 46 from each other to open the slit 48. In an insertion completion state in which the connector 100 is fitted with the catheter hub 20 (fixing member 70), the nozzle portion 52 greatly separates the pair of inclined portions 46. The state of the catheter indwelling body 22 at this time is illustrated in FIG. 4A, and the position of the opening member 50A is set as a second position.

The connector 100 allows a liquid (an infusion solution, blood, or the like) to flow into the catheter indwelling body 22 from the flow path 100a in the insertion completion state in which the connector 100 is tapered and fitted with the fixing member 70. Such a medicinal liquid flows in the distal direction through the flow passage 52a of the opening member 50A, flows out from the distal opening 52a1 of the nozzle portion 52 to the distal side of the valve body 40, and further flows into the lumen 12a of the catheter 12 from the distal-side space 36. The medicinal liquid that has flowed into the lumen 12a is administered through the distal opening 12a1 of the catheter 12 inserted into the patient's blood vessel.

When the connector 100 is removed after the administration, the connector 100 is displaced in the proximal direction relative to the catheter hub 20. At this time, the protrusion 62 (engaging portion 60) of the opening member 50A is engaged with the distal-side outer peripheral surface 102, and thus, is displaced following retraction of that the connector 100. That is, when the opening member 50A is pulled out in the valve body 40, the valve main body 44 is elastically restored to close the slit 48. Therefore, the valve body 40 inhibits the liquid in the distal-side space 36 from leaking to the proximal side.

When the opening member 50A retracts to the first position of the proximal-side space 38, the proximal tubular portion 54 is caught by the distal end of the fixing member 70. Because such catching is stronger than an engagement force by which the protrusion 62 (engaging portion 60) engages with the connector 100, the engagement between the protrusion 62 and the connector 100 is released. As a result, only the connector 100 is detached from the catheter hub 20.

In addition, the opening member 50A can open the valve body 40 by the same operation as described above when an infusion or a blood transfusion is performed again in the catheter indwelling body 22. That is, the catheter assembly 10A can suppress the leakage of the liquid while performing a plurality of infusions and blood transfusions with the operations of the valve body 40 and the opening member 50A.

Incidentally, the present invention is not limited to the above-described embodiment, and various modifications can be made in accordance with a gist of the invention. For example, the engaging portion 60 is not limited to being provided on the side wall portion 58 of the opening member 50A. As an example, the engaging portion 60 may be provided on the end wall portion 56 with which a distal surface of the connector 100 comes into contact.

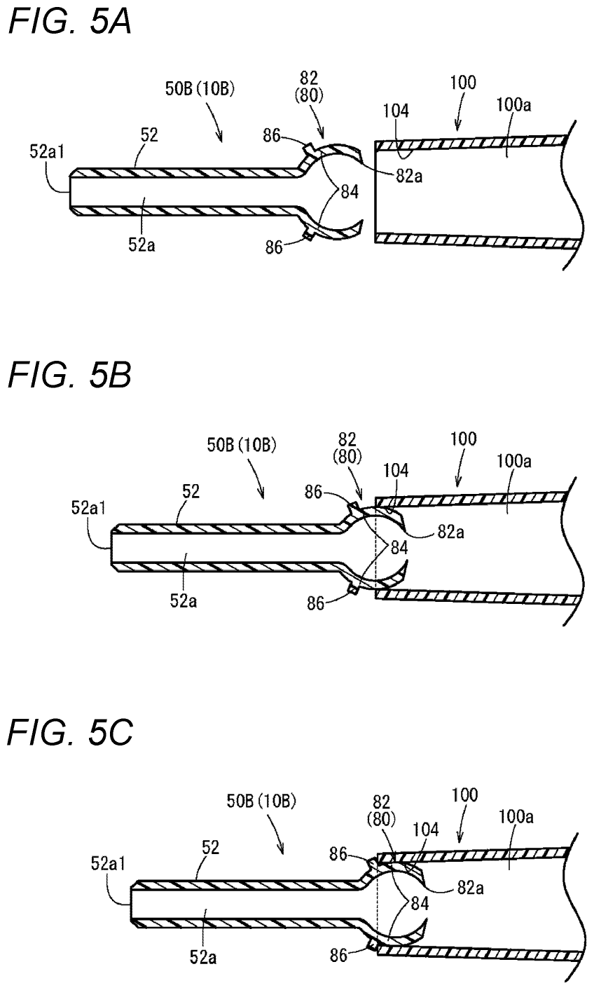

Hereinafter, some embodiments will be described in detail with reference to FIGS. 5A to 7C. Incidentally, an element having the same configuration or the same function as that in the above-described embodiment will be denoted by the same reference sign, and the detailed description thereof will be omitted in the following description.

### Second Embodiment

A catheter assembly 10B according to a second embodiment is different from the catheter assembly 10A in terms of including an opening member 50B having an engaging portion 80 that can engage with a distal-side inner peripheral surface 104 of the connector 100 as illustrated in FIGS. 5A to 5C.

For example, the opening member 50B is configured such that a proximal tubular portion 82 continuous with the proximal end of the nozzle portion 52 can be inserted into the flow path 100a of the connector 100 and can be elastically deformed, and thus, the entire proximal tubular portion 82 functions as the engaging portion 80. A diameter of the proximal tubular portion 82 once increases radially outward from a distal end in the proximal direction, and decreases radially inward from a substantially intermediate position in the axial direction in the proximal direction.

A site where the proximal tubular portion 82 protrudes radially outward the most is located on the radially outer side of the diameter of the flow path 100a of the connector 100 in the initial state in which the connector 100 is not inserted, and forms a side wall portion 84 that can come into contact with the distal-side inner peripheral surface 104 of the connector 100. The side wall portion 84 can be elastically displaced radially inward with the elastic deformation of the proximal tubular portion 82, and engages with the distal-side inner peripheral surface 104 with an appropriate frictional force when the proximal tubular portion 82 enters the flow path 100a of the connector 100.

On the other hand, a most proximal end 82a of the proximal tubular portion 82 that protrudes in the proximal direction the most is located on the radially inner side of the diameter of the flow path 100a of the connector 100. Therefore, the connector 100 can guide the most proximal end 82a to the flow path 100a when the connector 100 advances.

In addition, a projecting portion 86 that protrudes radially outward is provided to be closer to the distal side than the side wall portion 84 on an outer peripheral surface of the proximal tubular portion 82. The projecting portion 86 comes into contact with the distal end of the connector 100, thereby regulating the connector 100 from moving in the distal direction with respect to the opening member 50B. The projecting portion 86 is caught by the fixing member 70 when the connector 100 is retracted, thereby releasing the engagement between the proximal tubular portion 82 and the connector 100.

The opening member 50B of the catheter assembly 10B according to the second embodiment is basically configured as described above. The catheter assembly 10B causes the side wall portion 84 and the connector 100 to engage with each other as the connector 100 is inserted into the catheter hub 20 (see FIG. 4A).

That is, the most proximal end 82a enters the flow path 100a when the connector 100 advances in the distal direction. When the connector 100 further advances, a most distal end of the connector 100 passes over the side wall portion 84 by relatively moving in the distal direction while elastically deforming the proximal tubular portion 82 with respect to the opening member 50B that receives the resistance from the inner wall 20a of the catheter hub 20 and the valve body 40. When this most distal end passes over the side wall portion 84, the proximal tubular portion 82 is slightly elastically restored so as to expand radially outward, and the side wall portion 84 and the distal-side inner peripheral surface 104 engage with each other with an appropriate engaging force (frictional force). When further advancing, the connector 100 can come into contact with the projecting portion 86 to reliably push out the opening member 50B.

Therefore, when the nozzle portion 52 penetrates the valve body 40 (see FIG. 4A), the liquid supplied from the connector 100 favorably flow in the distal direction through the flow passage 52a. When the connector 100 retracts, the opening member 50B can also dependently retract on the basis of the engagement of the engaging portion 80, and come out of the valve body 40. Therefore, the catheter assembly 10B according to the second embodiment can also obtain the same effects as the catheter assembly 10A. Incidentally, the engaging portion 80 is not limited to the configuration of engaging with the distal-side inner peripheral surface 104 of the connector 100 by the frictional force (elastic force), and it is a matter of course that a configuration in which the engagement is achieved by biting, suction, adhesion, or the like an be adopted.

### Third Embodiment

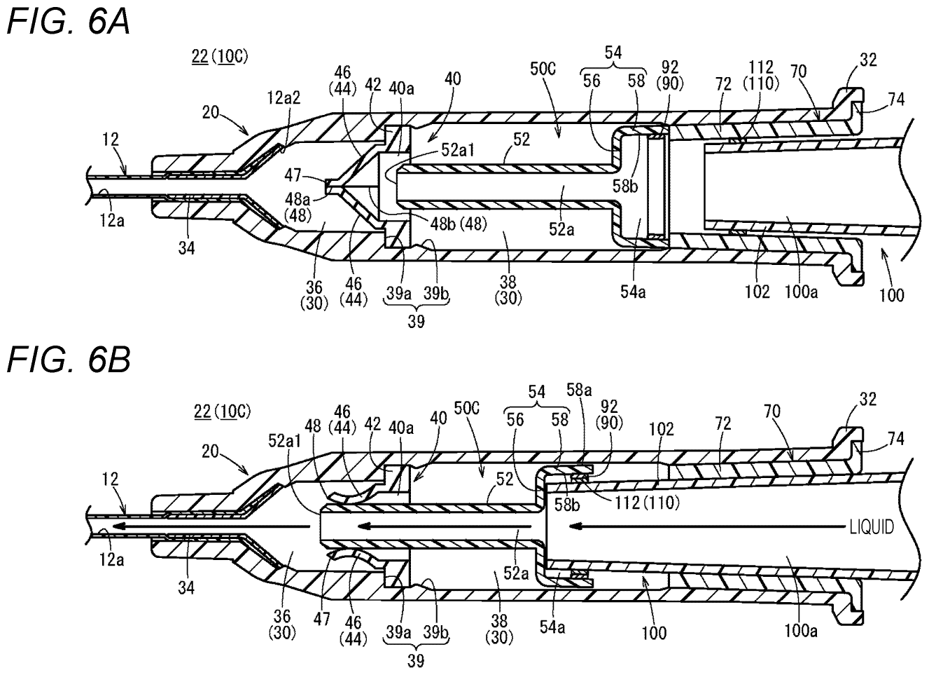

A catheter assembly 10C according to a third embodiment is different from the above catheter assemblies 10A and 10B in terms of providing an engaging portion 90 in an opening member 50C as illustrated in FIGS. 6A and 6B and providing an engaged portion 110 that can be engaged with the engaging portion 90 in the connector 100.

Specifically, as a first configuration example of the present embodiment, the opening member 50C includes a fastener portion 92, formed into a planar shape as the engaging portion 90, in the proximal tubular portion 54. The fastener portion 92 is provided, for example, over the entire circumference of the inner peripheral surface 58b of the proximal tubular portion 54 (side wall portion 58) in the circumferential direction, and a large number of minute hooks are raised on the inner surface thereof.

The connector 100 includes a joining portion 112, formed into a planar shape as the engaged portion 110, on the distal-side outer peripheral surface 102. The joining portion 112 is also provided, for example, over the entire circumference of the distal-side outer peripheral surface 102 in the circumferential direction, and a large number of minute loops are raised on the outer surface thereof. Incidentally, the fastener portion 92 and the joining portion 112 are not necessarily provided on the entire circumference in the circumferential direction, but may be provided on a part of the circumferential direction.

The above catheter assembly 10C can allow the opening member 50C and the connector 100 to be more smoothly engaged with each other with an appropriate engaging force by the engaging portion 90 (fastener portion 92) and the engaged portion 110 (joining portion 112). Therefore, when the connector 100 is retracted, the opening member 50C can be made to favorably follow to close the valve body 40.

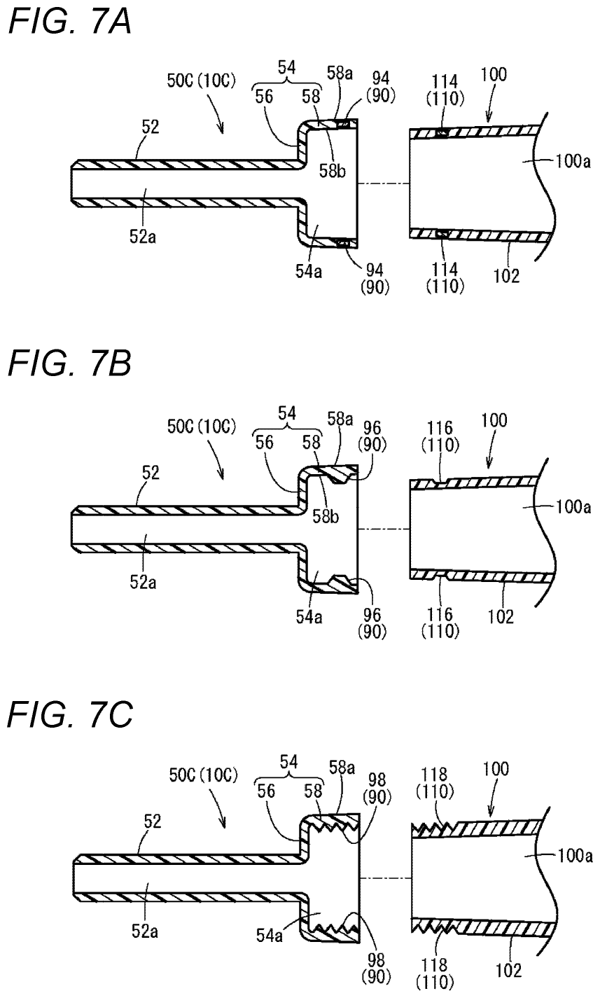

Incidentally, the engaging portion 90 and the engaged portion 110 are not limited to the above structures, and may adopt various structures. For example, a first magnetic body 94 can be applied as the engaging portion 90, and a second magnetic body 114 that is attracted to the first magnetic body 94 can be applied as the engaged portion 110 as in a second configuration example of the third embodiment illustrated in FIG. 7A.

In addition, a protrusion 96 can be applied as the engaging portion 90, and a recessed portion 116 into which the protrusion 96 is inserted (fitted) can be applied as the engaged portion 110, for example, as in a third configuration example of the third embodiment illustrated in FIG. 7B. Incidentally, it is a matter of course that the recessed portion 116 may be applied as the engaging portion 90, and the protrusion 96 may be applied as the engaged portion 110.

In addition, a female screw portion 98 can be applied as the engaging portion 90, and a male screw portion 118 into which the female screw portion 98 is screwed can be applied as the engaged portion 110, as in a fourth configuration example of the third embodiment illustrated in FIG. 7C. Incidentally, the male screw portion 118 may be applied as the engaging portion 90, and the female screw portion 98 may be applied as the engaged portion 110 in a configuration in which the opening member 50C is inserted into the distal-side inner peripheral surface 104 of the connector 100.

Incidentally, the catheter assemblies 10A to 10C may be formed by applying a plurality of types of the above-described configurations of the engaging portions 60, 80, and 90 and the engaged portion 110.

Technical ideas and effects that can be grasped from the above-described embodiments are described as follows.

The catheter assemblies 10A to 10C are provided with engaging portions 60, 80, and 90 on the opening members 50A to 50C, so that the opening members 50A to 50C and the connector 100 can be easily engaged with each other as the connector 100 advances. That is, the engaging portions 60, 80, and 90 can open the valve body 40 by integrating the opening members 50A to 50C and the connector 100 without requiring a large operating force. In addition, the catheter assemblies 10A to 10C can retract the opening members 50A to 50C following the connector 100 with the engagement of the engaging portions 60, 80, and 90 at the time of detaching the connector 100. As a result, the valve body 40 can be smoothly closed from the open state and the leakage of the liquid can be suppressed.

In addition, the engaging portions 60, 80, and 90 are provided on the tubular side wall portion 58 or 84 formed at the proximal ends of the opening members 50A to 50C. Thereby, the catheter assemblies 10A to 10C can favorably engage the connector 100 with the side wall portion 58 or 84 having the engaging portions 60, 80, or 90.

In addition, the side wall portion 58 is configured to allow the connector 100 to be inserted inside, and the engaging portions 60 and 90 can engage with the distal-side outer peripheral surface 102 of the connector 100. As a result, the engaging portions 60 and 90 can smoothly engage with the distal-side outer peripheral surface 102 when the connector 100 is inserted.

Alternatively, the side wall portion 84 is configured to be inserted into the flow path 100a of the connector 100, and the engaging portion 80 can engage with the distal-side inner peripheral surface 104 of the connector 100. As a result, the engaging portion 80 can smoothly engage with the distal-side inner peripheral surface 104 when the connector 100 is inserted.

In addition, the opening member 50A is formed harder than the connector 100, and the engaging portion 60 is the protrusion 62 capable of forming the anchor with respect to the connector 100. As a result, the protrusion 62 of the engaging portion 60 is firmly caught on the connector 100, and can favorably maintain the engagement state when the opening member 50A is retracted from the valve body 40.

In addition, the opening member 50A is configured to be softer than the connector 100, and the engaging portion 60 engages with the connector 100 by any one of a frictional force, suction, and adhesion. As a result, the engaging portion 60 can engage with the connector 100 with a sufficient engaging force.

In addition, the engaging portion 90 engages with the engaged portion 110 provided on the connector 100. As a result, the engaging portion 90 and the engaged portion 110 can be more easily engaged when the connector 100 is inserted.

In addition, the engaging portion 90 and the engaged portion 110 are engaged with each other by any one of a frictional force, a magnetic coupling force, fitting, and screwing. As a result, the engaging portion 90 and the engaged portion 110 are engaged with each other with a strong engaging force, and the opening member 50C and the connector 100 can be integrated.

In addition, the engaging portions 60, 80, and 90 allow the connector 100 to separate from the opening members 50A to 50C when the opening members 50A to 50C move to the first position as the connector 100 is retracted in the proximal direction. As a result, the catheter assemblies 10A to 10C can leave the opening members 50A to 50C in the catheter hub 20 when the connector 100 is detached, and can be favorably used a plurality of times.

The catheter hub 20 has the regulating portion (fixing member 70) that regulates the movement of the opening members 50A to 50C in the proximal direction in the state in which the opening members 50A to 50C are located at the first position. As a result, the catheter assemblies 10A to 10C can smoothly separate the connector 100 from the opening members 50A to 50C.

What is claimed is:

1. A catheter assembly comprising:
a catheter;
a catheter hub provided at a proximal end of the catheter;
a valve body fixed in the catheter hub; and
an opening member housed in the catheter hub to open the valve body;
wherein the opening member is movable between a first position proximal of the valve body and a second position for opening the valve body by being advanced by a connector inserted into the catheter hub; and
wherein the opening member comprises an engaging portion that engages with the connector with movement to the second position caused by the connector, and maintains the engagement with the connector and retracts the opening member from the valve body to the first position when the connector is retracted in a proximal direction and detached from the catheter hub.

2. The catheter assembly according to claim 1, wherein:
the engaging portion is provided on a tubular side wall portion formed at a proximal end of the opening member.

3. The catheter assembly according to claim 2, wherein:
the side wall portion has a configuration that allows the connector to be inserted inside; and
the engaging portion is engageable with a distal-side outer peripheral surface of the connector.

4. The catheter assembly according to claim 2, wherein:
the side wall portion is configured to be inserted into a flow path of the connector; and
the engaging portion is engageable with a distal-side inner peripheral surface of the connector.

5. The catheter assembly according to claim 1, wherein:
the opening member is harder than the connector; and
the engaging portion comprises a protrusion configured to anchor to the connector.

6. The catheter assembly according to claim 1, wherein:
the opening member softer than the connector; and
the engaging portion engages with the connector by a frictional force.

7. The catheter assembly according to claim 1, wherein:
the opening member softer than the connector; and
the engaging portion engages with the connector by suction.

8. The catheter assembly according to claim 1, wherein:
the opening member softer than the connector; and
the engaging portion engages with the connector by adhesion.

9. The catheter assembly according to claim 1, wherein:
the engaging portion engages with an engaged portion of the connector.

10. The catheter assembly according to claim 9, wherein:
the engaging portion and the engaged portion engage with each other by a frictional force, a magnetic coupling force, fitting, or screwing.

11. The catheter assembly according to claim 1, wherein:
the engaging portion allows the connector to separate from the opening member when the opening member moves to the first position as the connector is retracted in the proximal direction.

12. The catheter assembly according to claim 11, wherein:
the catheter hub has a regulating portion that regulates the movement of the opening member in the proximal direction in a state in which the opening member is located at the first position.

13. A catheter assembly comprising:
a catheter;
a catheter hub provided at a proximal end of the catheter;
a valve body fixed in the catheter hub; and
an opening member housed in the catheter hub to open the valve body, the opening member comprising a cylindrical nozzle portion and a proximal tubular portion;
wherein the opening member is movable between a first position proximal of the valve body and a second position in which the opening member is advanced by a connector inserted into the catheter hub and the cylindrical nozzle opens the valve body; and
wherein the opening member comprises an engaging portion that engages with the connector with movement to the second position caused by the connector, and maintains the engagement with the connector and retracts the opening member from the valve body to the first position when the connector is retracted in a proximal direction and detached from the catheter hub, wherein the engaging portion comprises a protrusion that protrudes at an inner peripheral surface of the proximal tubular portion.

14. A catheter assembly comprising:
a catheter;
a catheter hub provided at a proximal end of the catheter;
a valve body fixed in the catheter hub; and
an opening member housed in the catheter hub to open the valve body, the opening member comprising a cylindrical nozzle portion and a proximal portion;
wherein the opening member is movable between a first position proximal of the valve body and a second position in which the opening member is advanced by a connector inserted into the catheter hub and the cylindrical nozzle opens the valve body; and
wherein a diameter of the proximal portion of the opening member increases from a distal end of the proximal portion to an intermediate position of the proximal portion, and decreases from the intermediate position to a proximal end of the proximal portion; and
wherein the proximal portion of the opening member is configured to be inserted into a flow path of the connector and engage with a distal-side inner peripheral surface of the connector with movement to the second position caused by the connector, and to maintain the engagement with the connector and retract the opening member from the valve body to the first position when the connector is retracted in a proximal direction and detached from the catheter hub.
